(19) United States
(12) Patent Application Publication
(10) Pub. No.: US 2012/0022892 A1
(43) Pub. Date: Jan. 26, 2012

(54) ELECTRONIC MEDICAL RECORD INTERACTIVE INTERFACE SYSTEM

(75) Inventors: David Philip FELDMAN, Millbrae, CA (US); Joshua Howland TAMAYO-SARVER, Los Gatos, CA (US); Khoa That Hoa TON, San Jose, CA (US)

(73) Assignee: INTERFACEED SOLUTIONS, LLC, Millbrae, CA (US)

(21) Appl. No.: 13/187,394

(22) Filed: Jul. 20, 2011

Related U.S. Application Data
(60) Provisional application No. 61/366,135, filed on Jul. 20, 2010.
(60) Provisional application No. 61/421,169, filed on Dec. 8, 2010.

(51) Int. Cl. G06Q 50/00; A61B 5/00

(52) U.S. Cl. 705/3; 600/300

(57) ABSTRACT

A method of confirming a medical diagnosis using a graphical user interface includes receiving an input selecting a first medical diagnosis as a cause of a patient complaint, and displaying a template based on the first medical diagnosis. The template includes a first query associated with the first medical diagnosis, a suggested response to the first query, and a first input field configured to receive the suggested response to the first query. The method includes using provider-configurable diagnostic based templates facilitating automated data entry to current and future electronic medical records systems. Systems and machine-readable media are also described.

### CROSS-REFERENCE TO RELATED APPLICATIONS

The present application claims the benefit of priority under 35 U.S.C. §119 from U.S. Provisional Patent Application Ser. No. 61/366,135, filed Jul. 20, 2010; and U.S. Provisional Patent Application Ser. No. 61/421,169, filed Dec. 8, 2010, the disclosures of which are hereby incorporated by reference in their entirety for all purposes.

### BACKGROUND

1. Field

The present disclosure generally relates to electronic files, and specifically to the creation and editing of electronic medical records.

2. Description of the Related Art

Electronic medical records (“EMR”) (also known as electronic health records or electronic patient records) are used, in part, to create financial efficiencies and decrease administrative waste in an effort to address the overwhelming cost and currently identified inefficiencies in the United States healthcare system today. The current state of this effort, however, is fragmented and leaves patients and healthcare providers (e.g., physicians, physician assistants, nurse practitioners, registered nurses) frustrated and resistant to the EMR options available to them. Often, the choice of EMR systems is either mandated by the hospital or health system that a provider works in, or is limited by the EMR systems that are commercially available, financially affordable, and are a “best fit” for a given practice. In many cases, the experience with EMR is one that reduces productivity, requires a change in workflow, produces less useful clinical documentation, and has a cumbersome interface. As a result, providers are reluctant and resistant to using a system that requires more time and effort spent on documentation, especially given that it typically adds hours in increased work in a day and takes the provider away from direct patient care.

### SUMMARY

In certain embodiments of the present disclosure, a method of providing information on a medical diagnosis is disclosed. The method includes receiving, from a user, an input identifying the medical diagnosis, and requesting, from a server, a template based on the identified medical diagnosis. The template includes at least one query associated with the identified medical diagnosis, at least one suggested response to the at least one query, and an input field configured to receive the at least one suggested response to the at least one query. The at least one suggested response is configured to be selected by the user for entry into the input field. The method also includes receiving the template from the server, and displaying, on a display device, the template to the user, and generating a record based on the template and the at least one suggested response, by the user, to the at least one query.

In certain embodiments of the present disclosure, a system for providing information on a medical diagnosis is disclosed. The system includes a processor configured to request, from a server, a template based on the medical diagnosis, and to receive the template from the server, and a memory that includes the template. The template includes at least one query associated with the medical diagnosis, at least one suggested response to the at least one query, and an input field configured to receive the at least one suggested response to the at least one query. The at least one suggested response is configured to be selected by a user for entry into the input field. The processor is further configured to receive, from the user, an input identifying the medical diagnosis, to provide the template based on the identified medical diagnosis, and to generate a record based on the template and the at least one suggested response, by the user, to the at least one query.

In certain embodiments of the present disclosure, a machine-readable medium that includes machine-readable instructions for causing a processor to execute a method of providing information on a medical diagnosis is disclosed. The method includes receiving, from a user, an input identifying the medical diagnosis, and requesting, from a server, a template based on the identified medical diagnosis. The template includes at least one query associated with the identified medical diagnosis, at least one suggested response to the at least one query, and an input field configured to receive the at least one suggested response to the at least one query. The at least one suggested response is configured to be selected by the user for entry into the input field. The method also includes receiving the template from the server, and displaying, on a display device, the template to the user, and generating a record based on the template and the at least one suggested response, by the user, to the at least one query.

In certain embodiments of the present disclosure, a system for providing information on a medical diagnosis is disclosed. The system includes a processor configured to receive, from a client, a request for a template based on the medical diagnosis, and a memory that includes the template. The template includes at least one query associated with the medical diagnosis, at least one suggested response to the at least one query, and an input field configured to receive an actual response to the at least one query. The at least one suggested response is configured to be selected by a user for entry into the input field. The processor is further configured to provide, to the client, the template from the memory.

In certain embodiments of the present disclosure, a method of providing information on a medical diagnosis is disclosed. The method includes receiving, from a client, a request for a template based on the medical diagnosis, and retrieving, from a memory, the template. The template includes at least one query associated with the identified medical diagnosis, at least one suggested response to the at least one query, and an input field configured to receive an actual response to the at least one query. The at least one suggested response is configured to be selected by the user for entry into the input field. The method also includes providing, to the client, the template.

In certain embodiments of the present disclosure, a machine-readable medium that includes machine-readable instructions for causing a processor to execute a method of providing information on a medical diagnosis is disclosed. The method includes receiving, from a client, a request for a template based on the medical diagnosis, and retrieving, from a memory, the template. The template includes at least one query associated with the identified medical diagnosis, at least one suggested response to the at least one query, and an input field configured to receive an actual response to the at least one query. The at least one suggested response is configured to be selected by the user for entry into the input field. The method also includes providing, to the client, the template.

In certain embodiments of the present disclosure, a method of confirming a medical diagnosis using a graphical user interface is disclosed. The method includes receiving an input selecting a first medical diagnosis as a cause of a patient complaint, and displaying, on a display device, a template based on the first medical diagnosis. The template includes a first query associated with the first medical diagnosis, a suggested response to the first query, and a first input field configured to receive the suggested response to the first query. The template also includes a second query associated with a second medical diagnosis. The second medical diagnosis is an alternative to the first medical diagnosis. The template further includes a suggested response to the second query, and a second input field configured to receive the suggested response to the second query. The suggested response to the first query is configured to be selected by the user for entry into the first input field and the suggested response to the second query is configured to be selected by the user for entry into the second input field. The method also includes generating a record based on the template and a suggested response received by the first input field and a suggested response received by the second input field.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1A illustrates an exemplary architecture for providing information on a medical diagnosis according to certain embodiments.

FIGS. 1B-1, 1B-2, 1C-1, 1C-2, 1D-1, and 1D-2 are exemplary screenshots illustrating the association between a user interface of the disclosed system and a user interface of an underlying EMR system.

FIG. 2 is an exemplary process for providing information on a medical diagnosis using the architecture of FIG. 1A.

FIGS. 3A, 3B, 3C, 3D, 3E, 3F, 3G, 3H, 3I, 3J, 3K, 3L, 3M, 3N, 3O, 3P, and 3Q are exemplary screenshots that illustrate various steps for providing information on a medical diagnosis using the architecture of FIG. 1A.

FIG. 4 is a block diagram illustrating an example of a computer system with which the client, the local server, or the remote server illustrated in FIG. 1A can be implemented.

### DETAILED DESCRIPTION

In the following detailed description, numerous specific details are set forth to provide a full understanding of the present disclosure. It will be apparent, however, to one ordinarily skilled in the art that the embodiments of the present disclosure may be practiced without some of these specific details. In other instances, well-known structures and techniques have not been shown in detail not to obscure the disclosure.

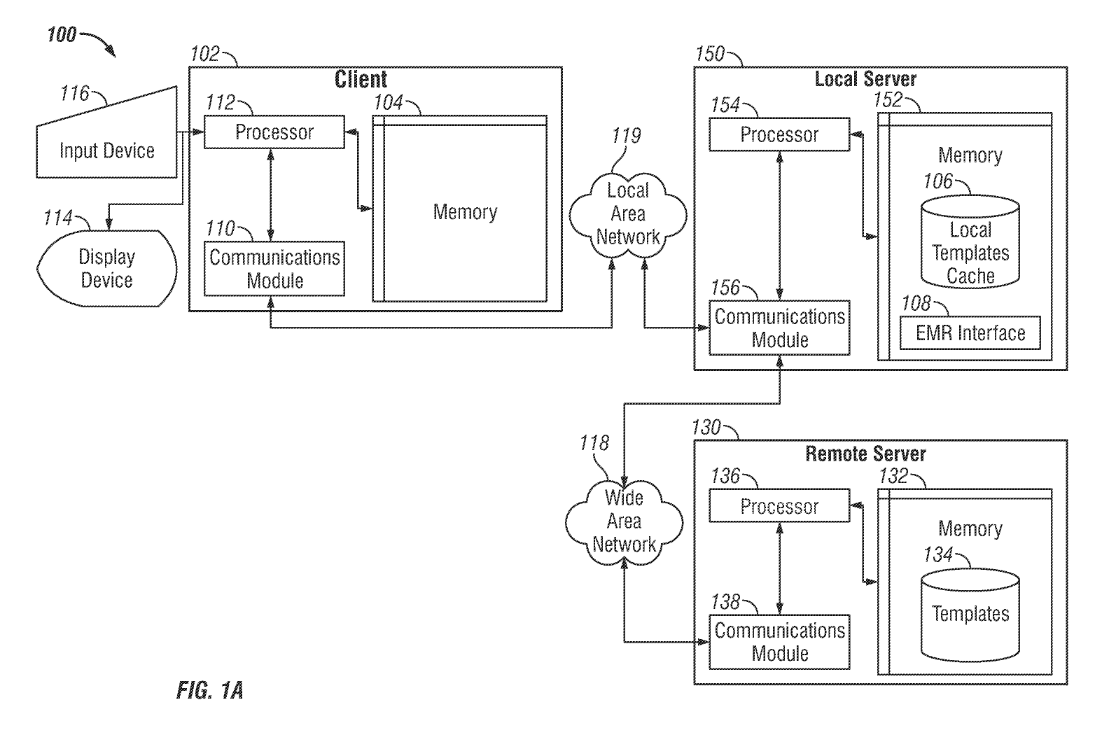

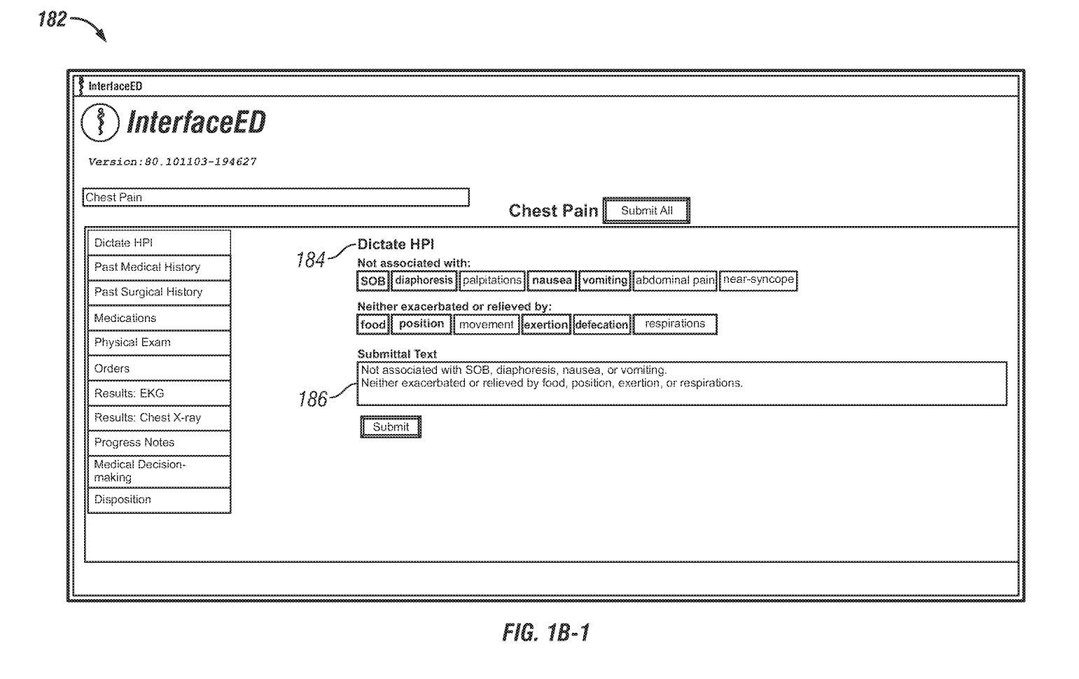

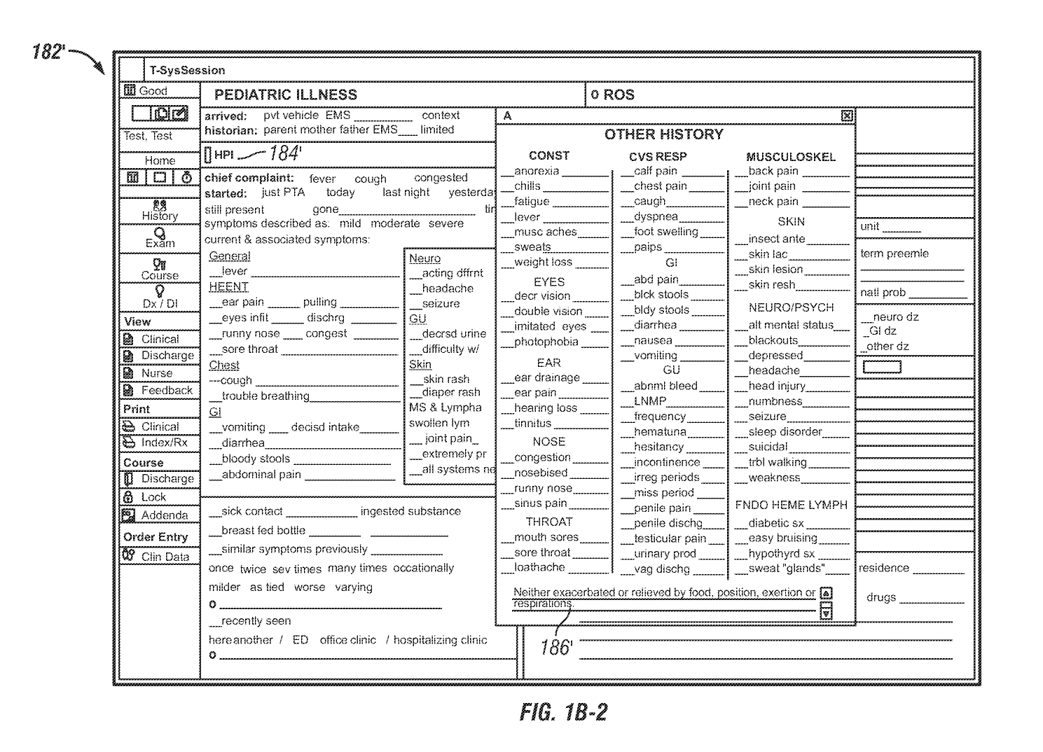

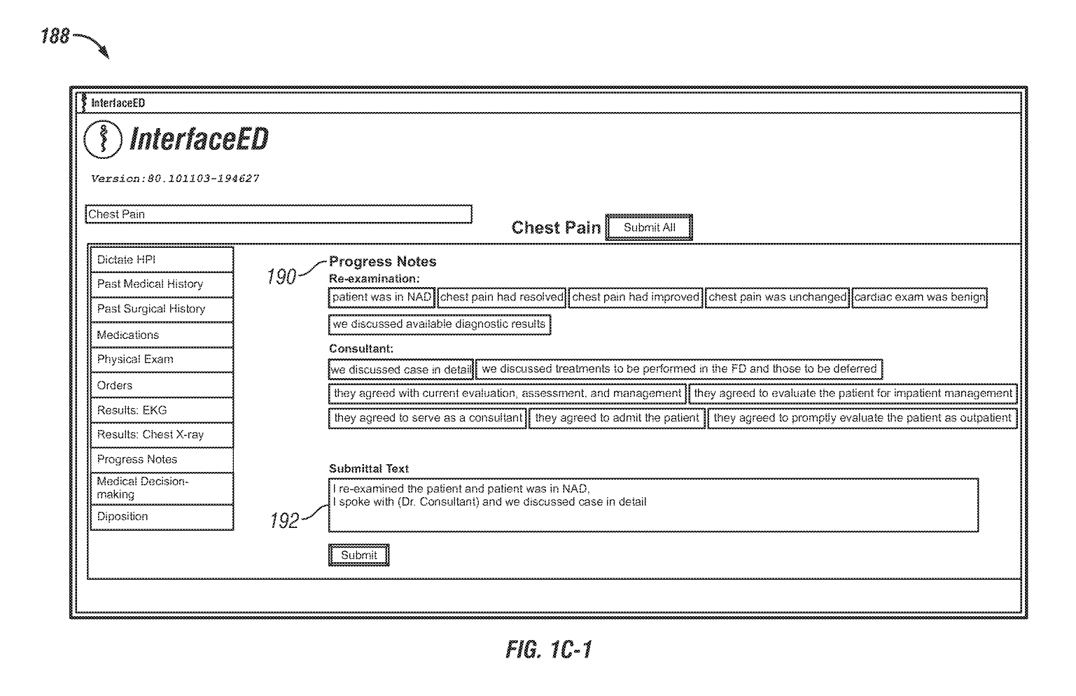

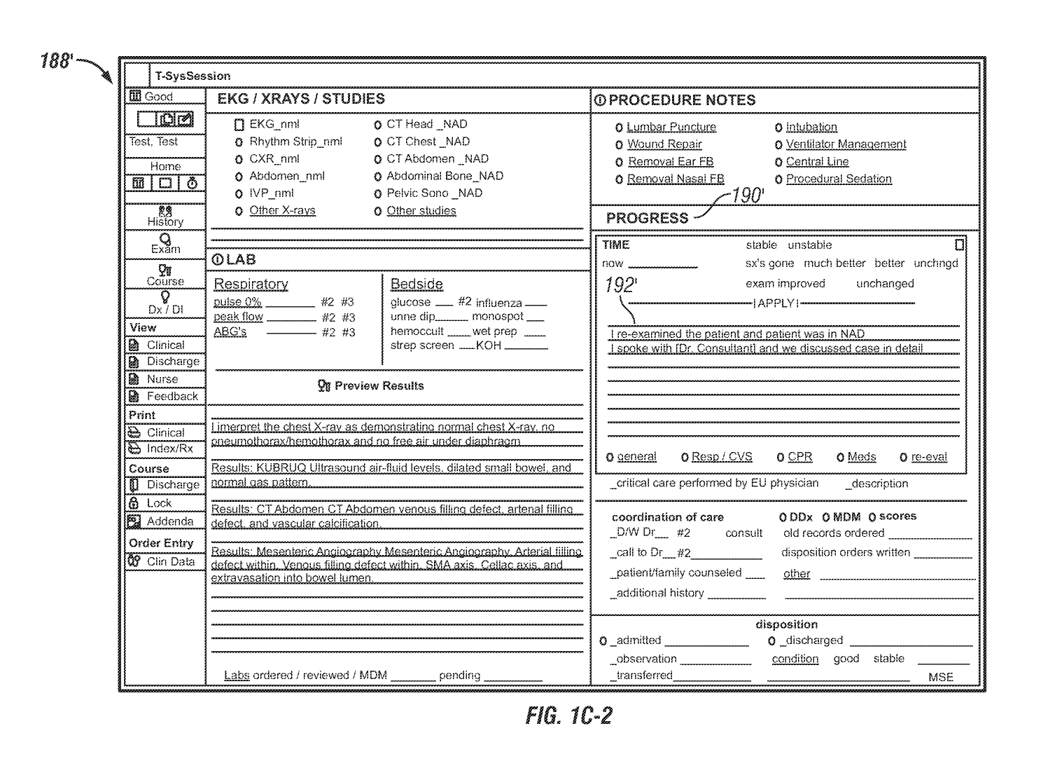

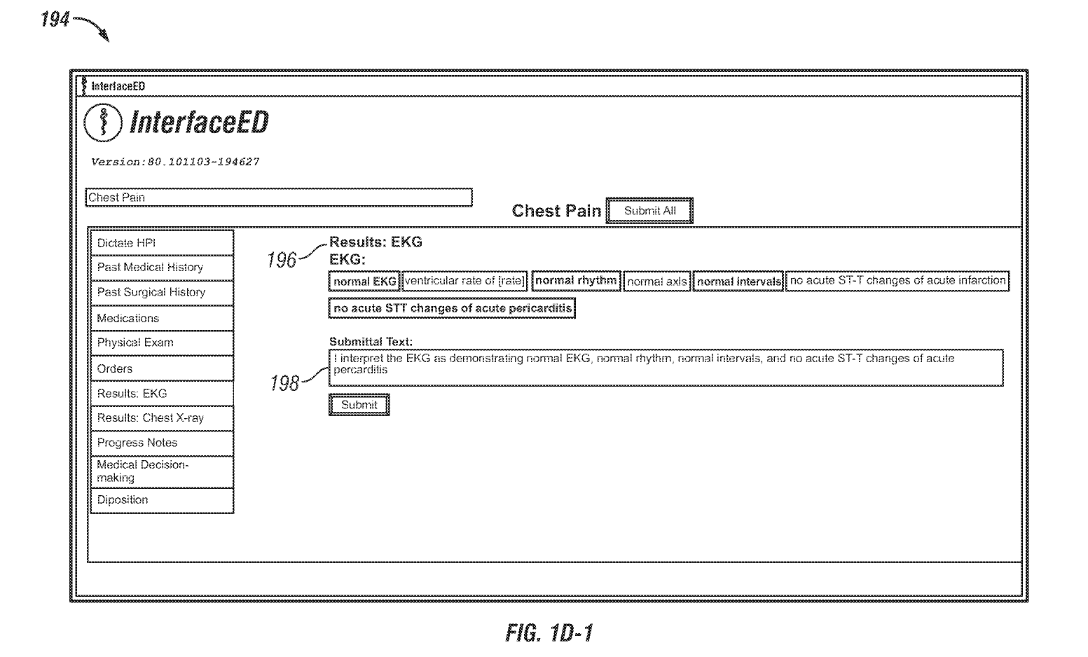

FIG. 1A illustrates an exemplary architecture for providing information on a medical diagnosis according to certain embodiments. The architecture 100 includes a client 102, a local server 150, and a remote server 130. The client 102 is connected to the local server 150 over a local area network 119, and the local server 150 is connected to the remote server 130 over a wide area network 118. The local area network 119 can be, for example, a private communications network, and the wide area network 118 can be, for example, the Internet.

The remote server 130 includes a processor 136, communications module 138, and memory 132, the memory 132 including a plurality of templates 134. The remote server 130 is configured for communication over the wide area network 118 (e.g., with local server 150) using a communications module 138. The communications module 138 can be, for example, a modem or Ethernet card. The processor 136 of the remote server 130 is configured to execute instructions, such as instructions physically coded into the processor 136, instructions received from software in memory 132, or a combination of both. For example, the processor 136 of the remote server 130 is configured to provide templates 134 to the local server 150, such as when the local server 150 sends a request for the templates 134 over the wide area network 118.

The local server 150 includes a processor 154, a communications module 156, and a memory 152, the memory including a local templates cache 106 and an EMR interface 108. The local templates cache 106 is configured to receive either a subset of the templates 134 stored on the remote server 130, or all of the templates stored on the remote server 130. In certain embodiments, the templates stored in the local templates cache 106 are updated with the templates 134 stored on the remote server 130. The processor 136 of the local server 150 is configured to execute instructions, such as instructions physically coded into the processor 136, instructions received from software in memory 132, or a combination of both.

The client 102 includes a processor 112, a communications module 110, a memory 104, an input device 116, and a display device 114. The processor 112 of the client 102 is configured to execute instructions, such as instructions physically coded into the processor 112, instructions received from software in memory, or a combination of both. For example, the processor 136 is configured to display a template, including its queries and suggested responses, using the display device 114, and is configured to receive selections of suggested responses or manual user input using the input device 116. Exemplary display devices 114 include CRT or LCD monitors, and exemplary input devices 116 include keyboards, mice, touch screens, trackballs, and microphones.

The templates 134 stored in either the memory 132 of the remote server 130 and/or the local templates cache 106 of the local server 150 are each based on at least one medical diagnosis. As discussed herein, a medical diagnosis is an identification of a medical disease or injury, or an identification of a complaint of a medical disease or injury. Exemplary medical diagnoses include, but are not limited to, chest pain, abdominal pain, back pain, pneumonia, appendicitis, diverticulitis, urinary tract infection, pyelonephritis, myocardial infarction, pulmonary embolus, aortic dissection, cerebral vascular accident, transient ischemic attack, meningitis, and encephalitis. In certain embodiments, a template may be temporarily stored in the memory 104 of the client 102 while it is displayed by the client. The templates are stored as data files. In certain embodiments, each template includes at least one query associated with the medical diagnosis, at least one suggested response to the query, and an input field configured to receive an actual response to the query. As discussed herein, a query includes, but is not limited to, a prompt indicating a response is requested, clinical and/or other pertinent information entered in the input fields by the user, clinical and/or other pertinent information entered in the input fields by the automated response of the program, diagnostic and/or therapeutic interventions as related to the diagnosis, critical decision making as related to the diagnosis, medical decision making as related to the diagnosis, satisficing of alternative diagnoses, discussion of further medical care and clinical disposition. The suggested response is configured to be selected by a user (e.g., provider) for entry into the input field. The suggested response is based on, for example, at least one of a previously provided response to a query (e.g., because much of the documentation required by a patient encounter is constant and predictable), a user-defined suggested response (e.g., what a user believes a suggested response to the query should be), a facility policy (e.g., a response in accordance with the policies of the healthcare facility of the user), and a best practice (e.g., in accordance with the best practices of the healthcare facility of the user or standard of practice as defined by a larger healthcare body, such as the American College of Emergency Physicians, American Board of Emergency Medicine, American Medical Association, California Medical Association and equivalent state medical associations, American Hospital Association, American College of Cardiology, Association of American Medical Colleges, Accreditation Council for Continuing Medical Education, Accreditation Council for Graduate Medical Education, American Academy of Neurology, American Academy of Pediatrics, American Academy of Orthopedic Surgeons, American College of Surgeons, American Dental Association, American Health Care Association, American Health Lawyers Association,). In certain embodiments, the suggested responses are based on user-defined suggested responses that are received from various clients 102 that are connected over the wide area network 118 (e.g., from different healthcare facilities that are subscribed to a service that includes providing the templates 134).

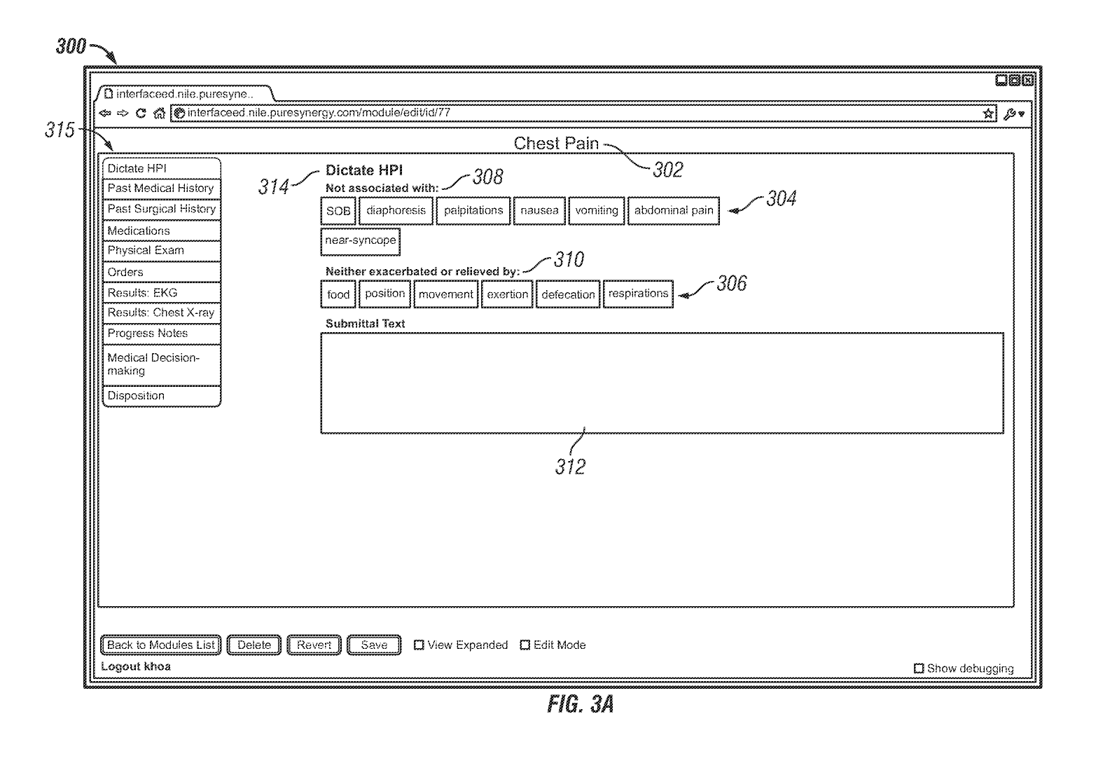

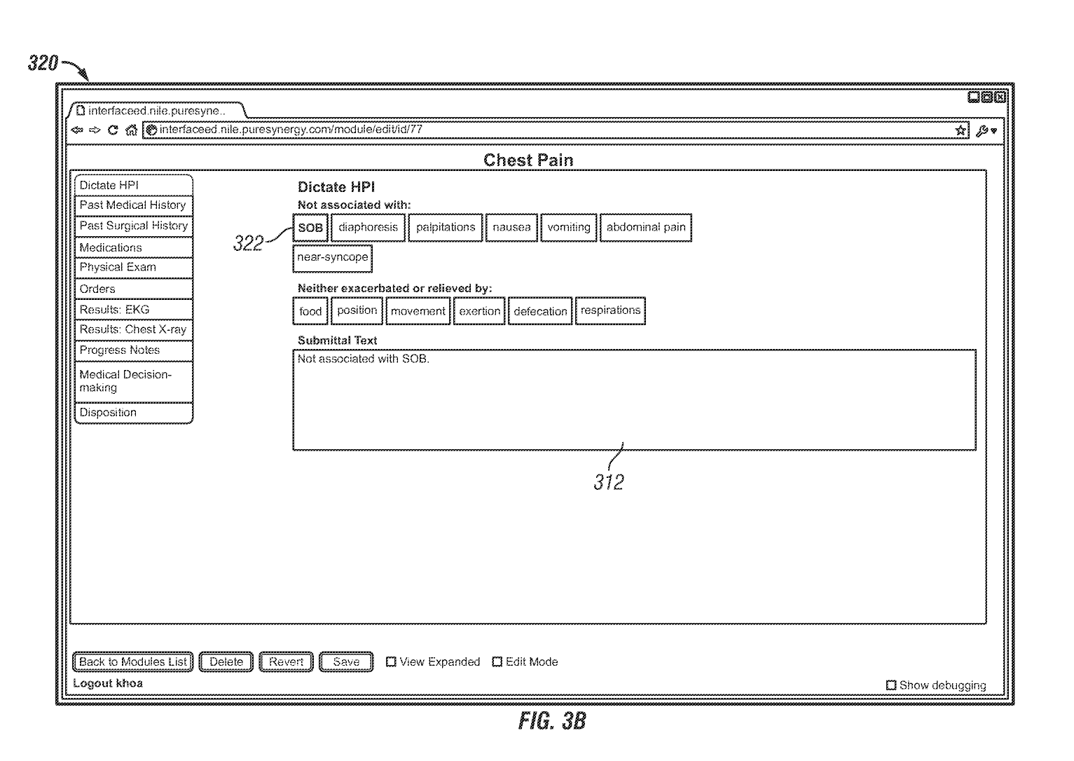

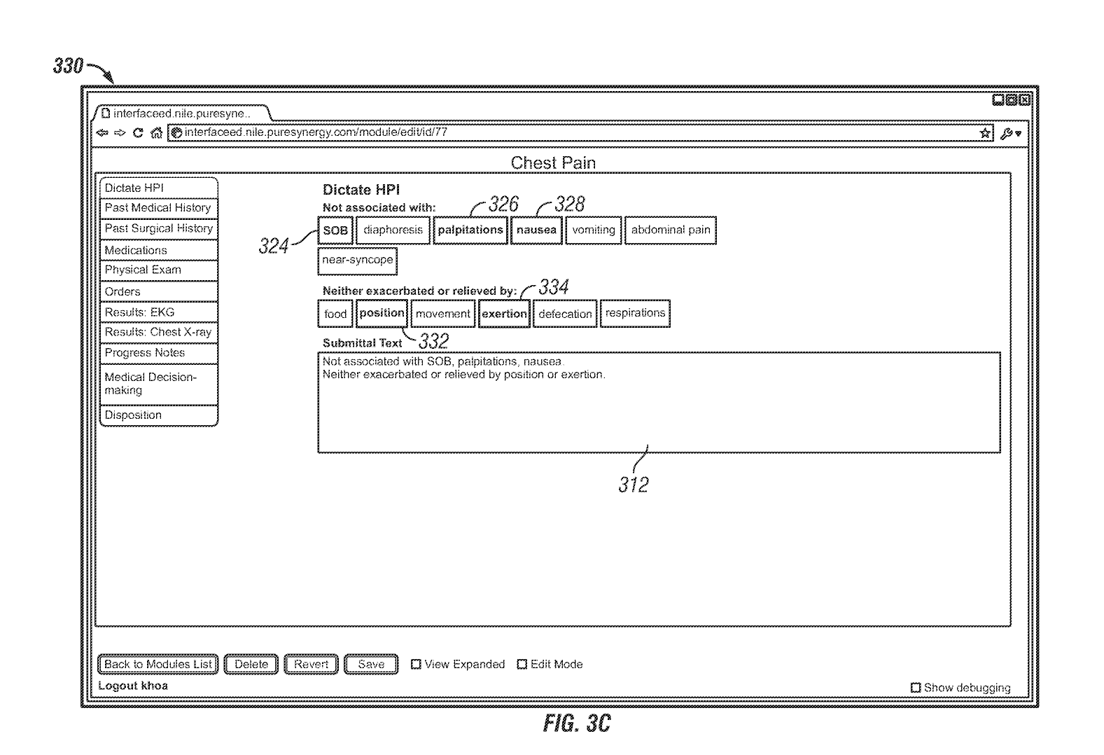

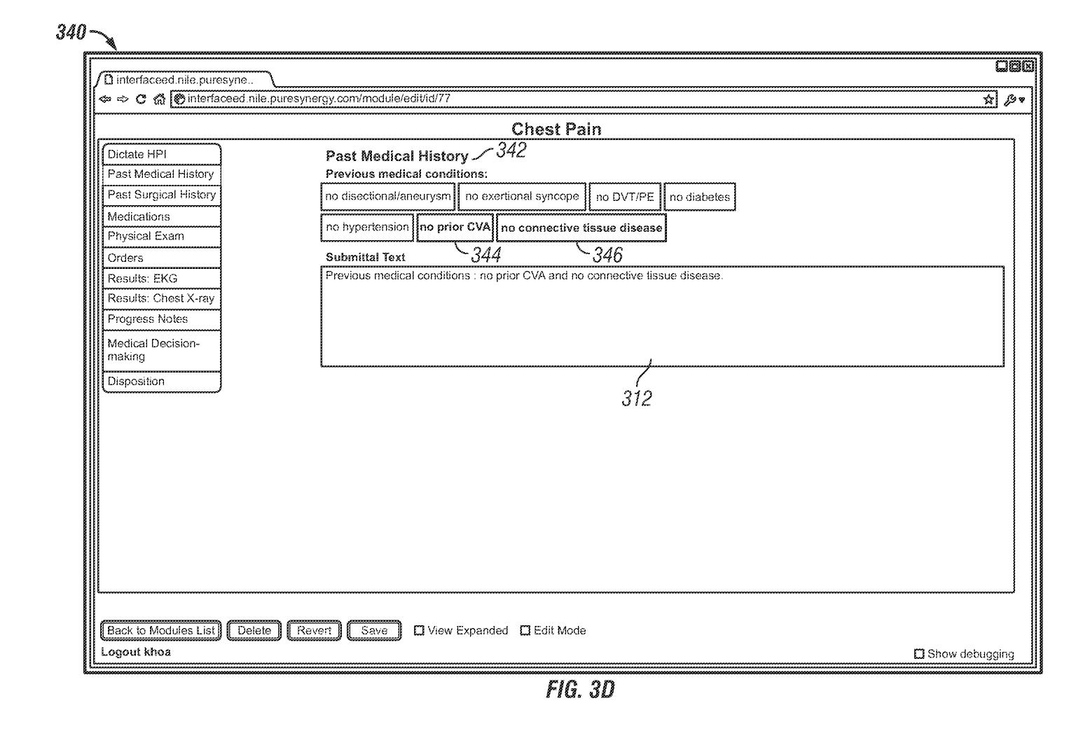

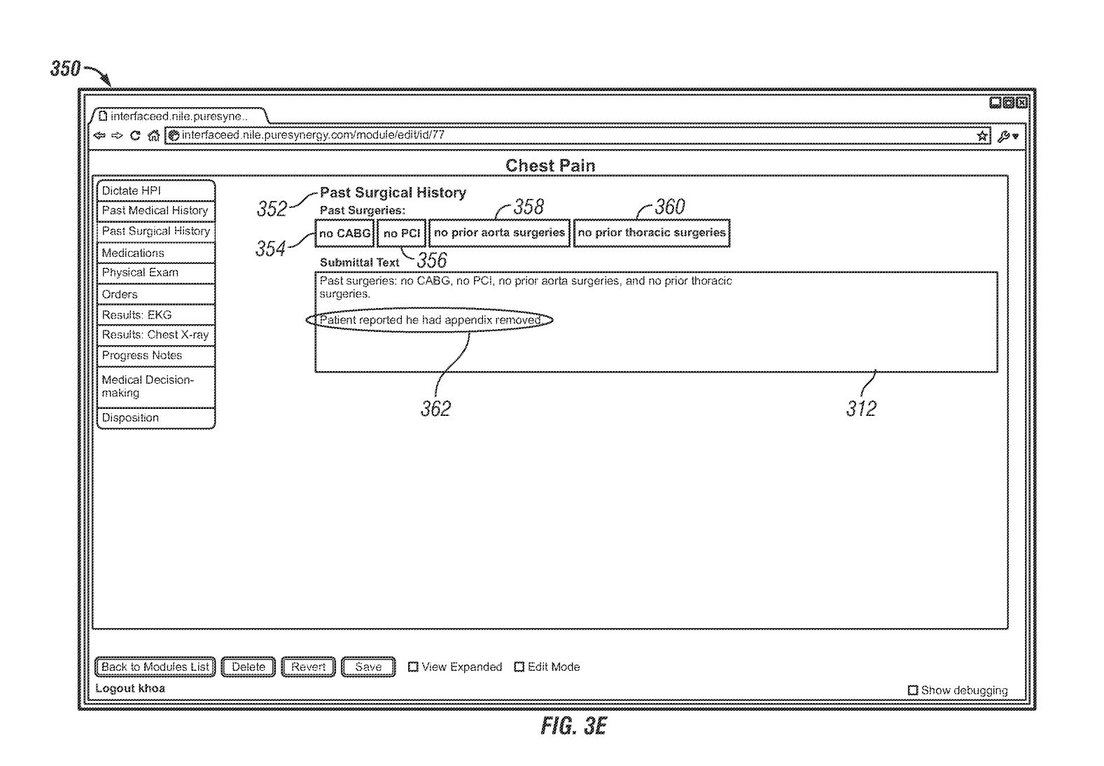

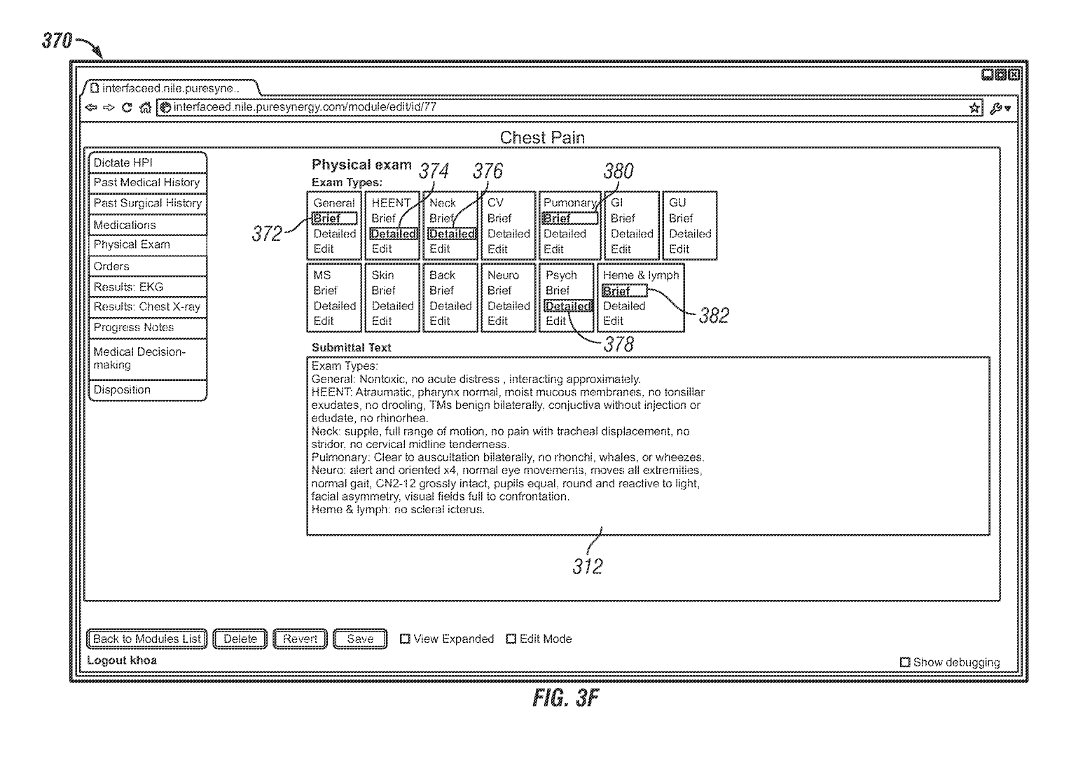

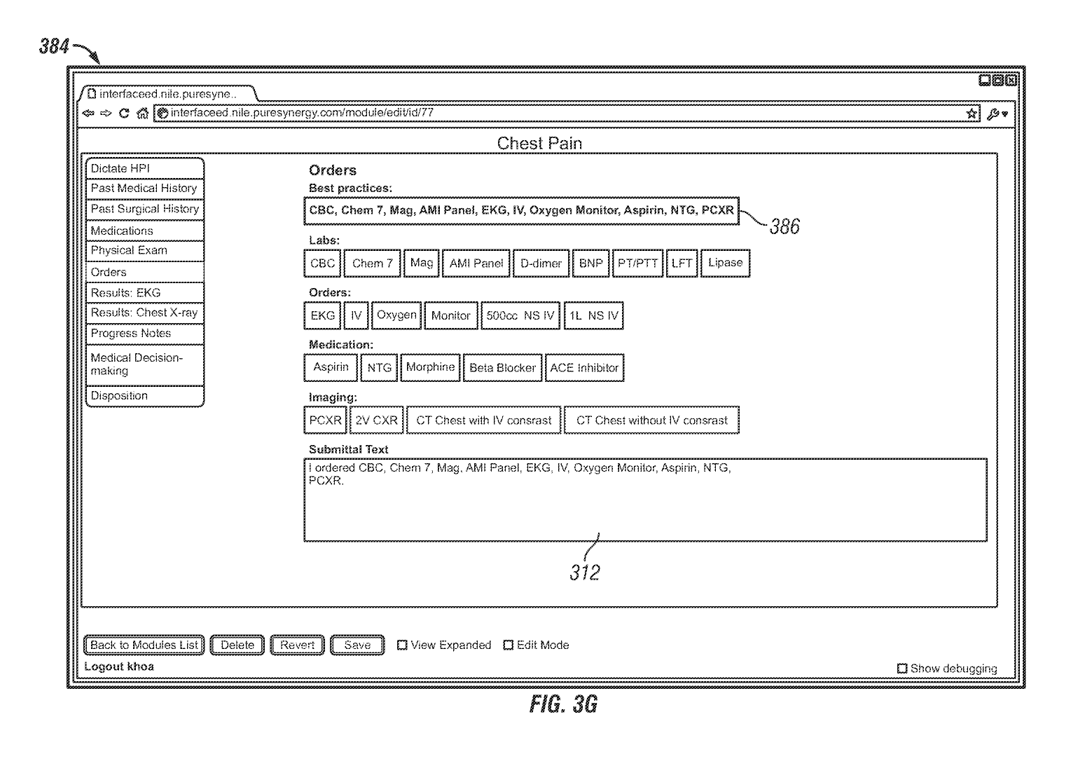

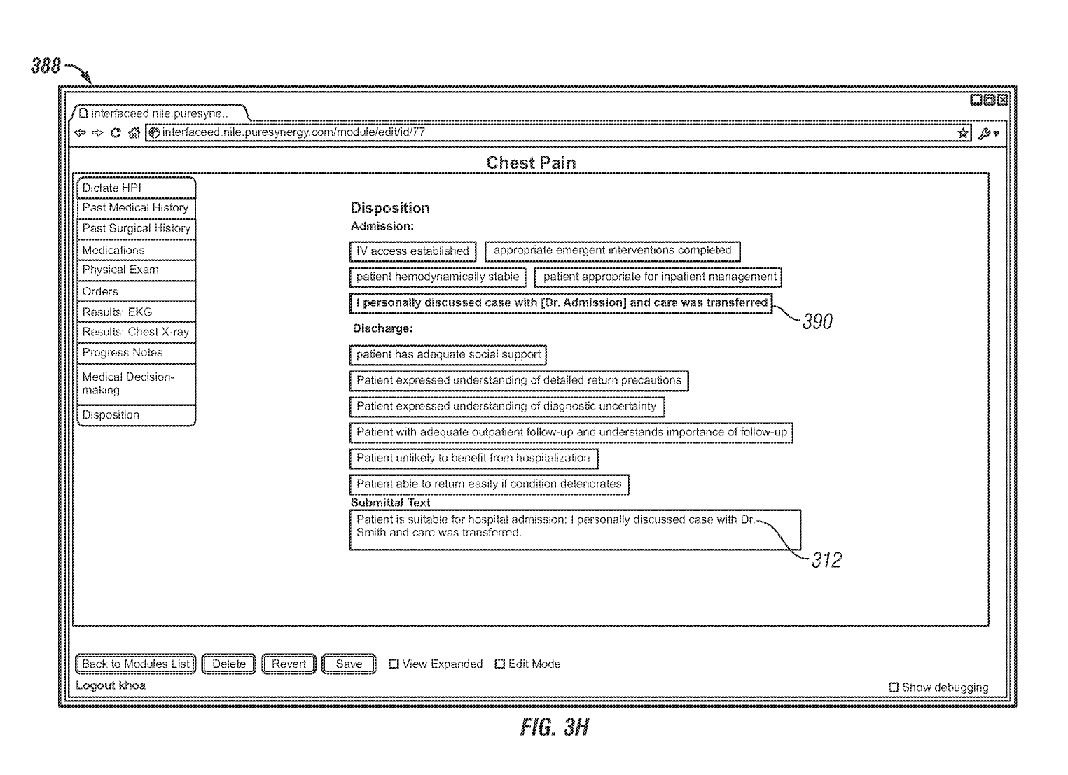

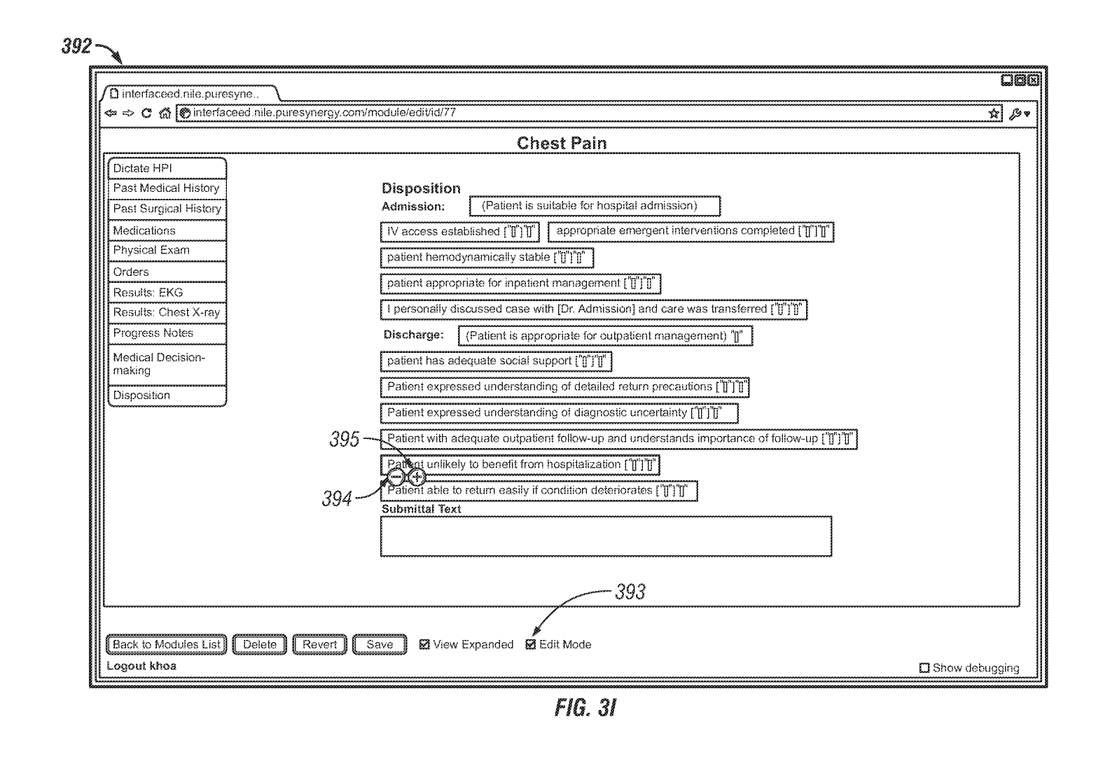

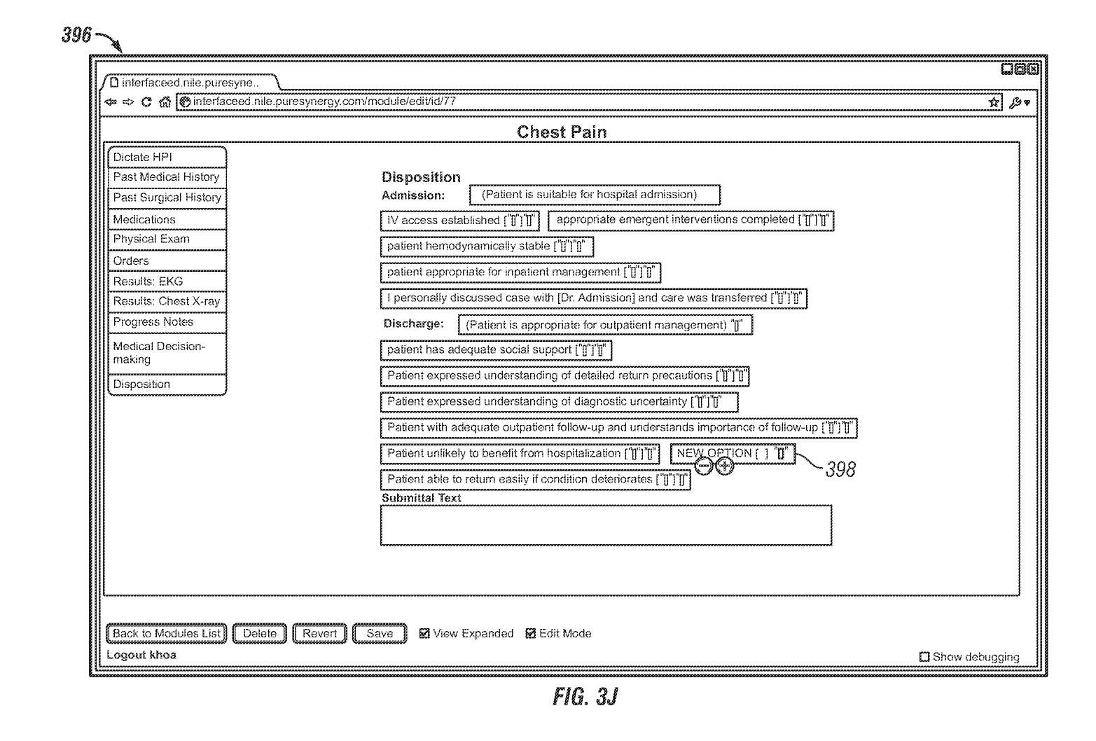

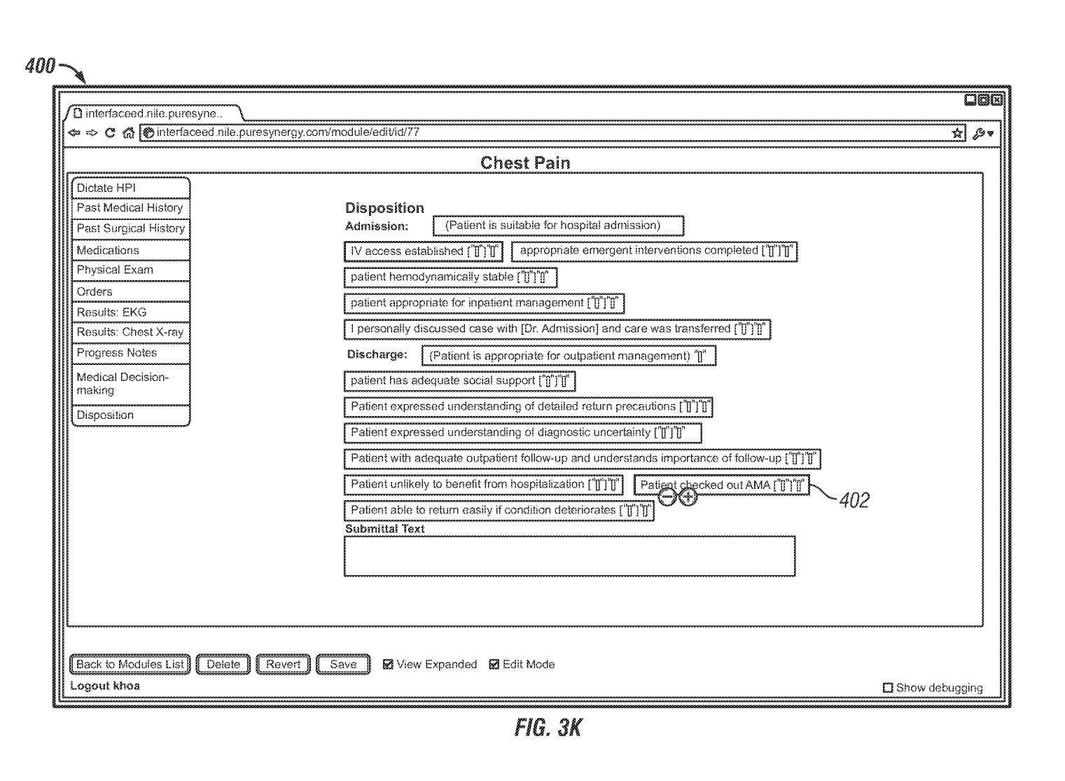

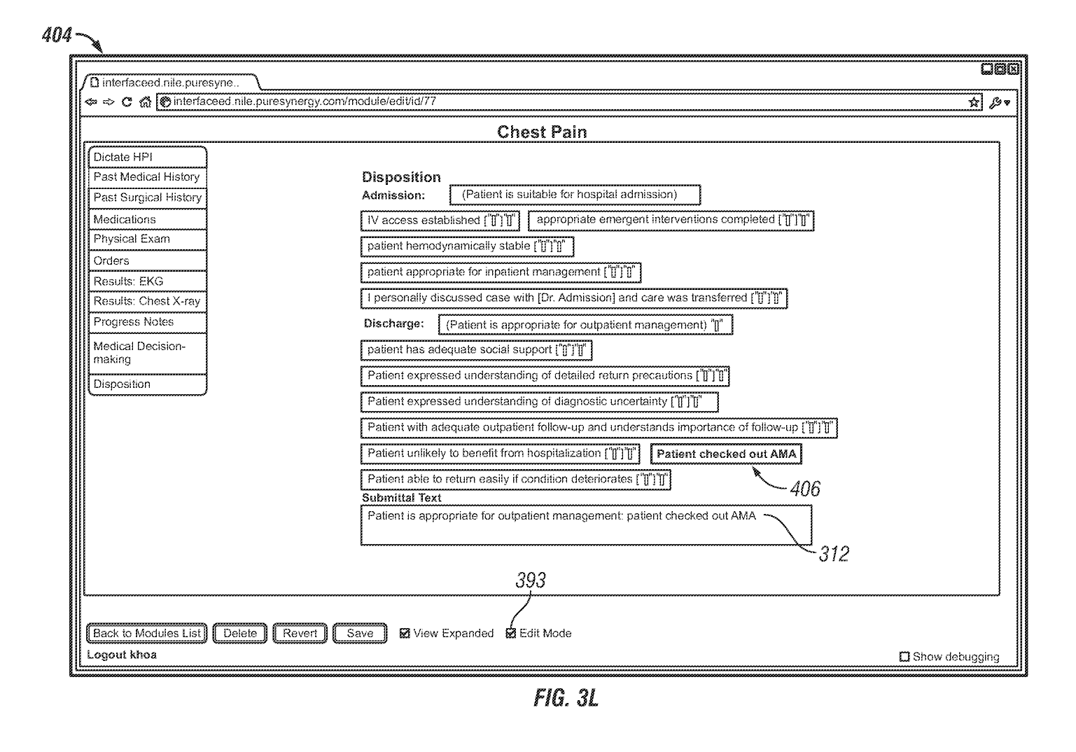

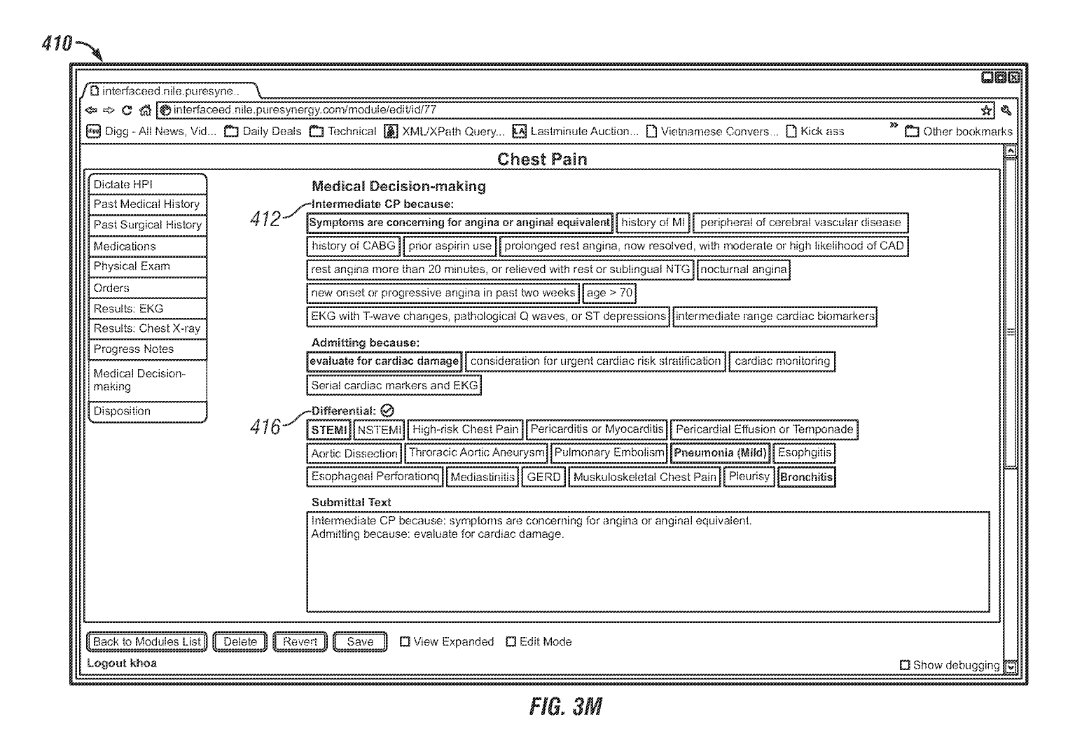

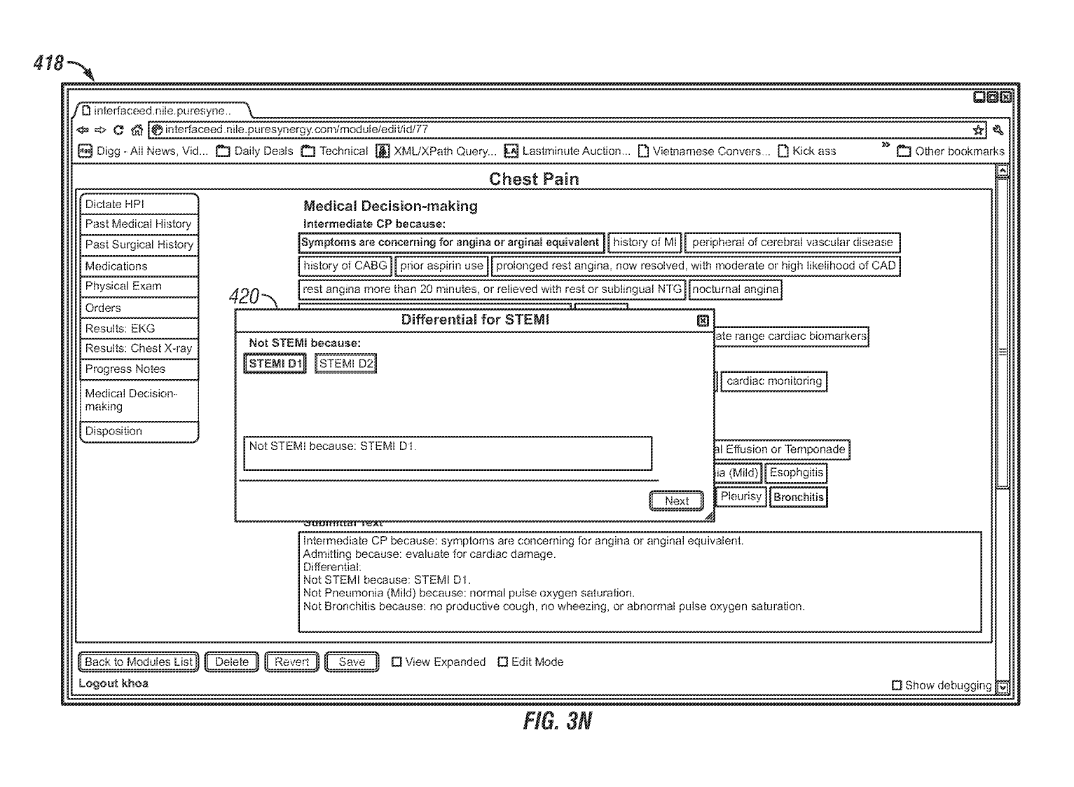

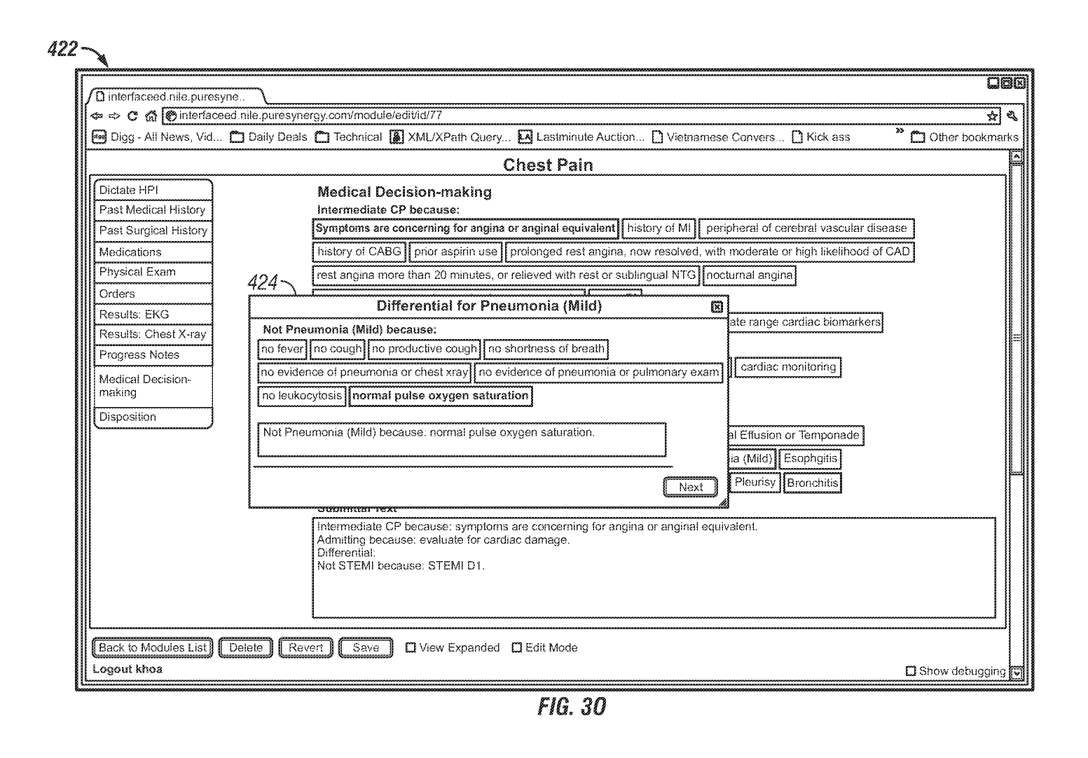

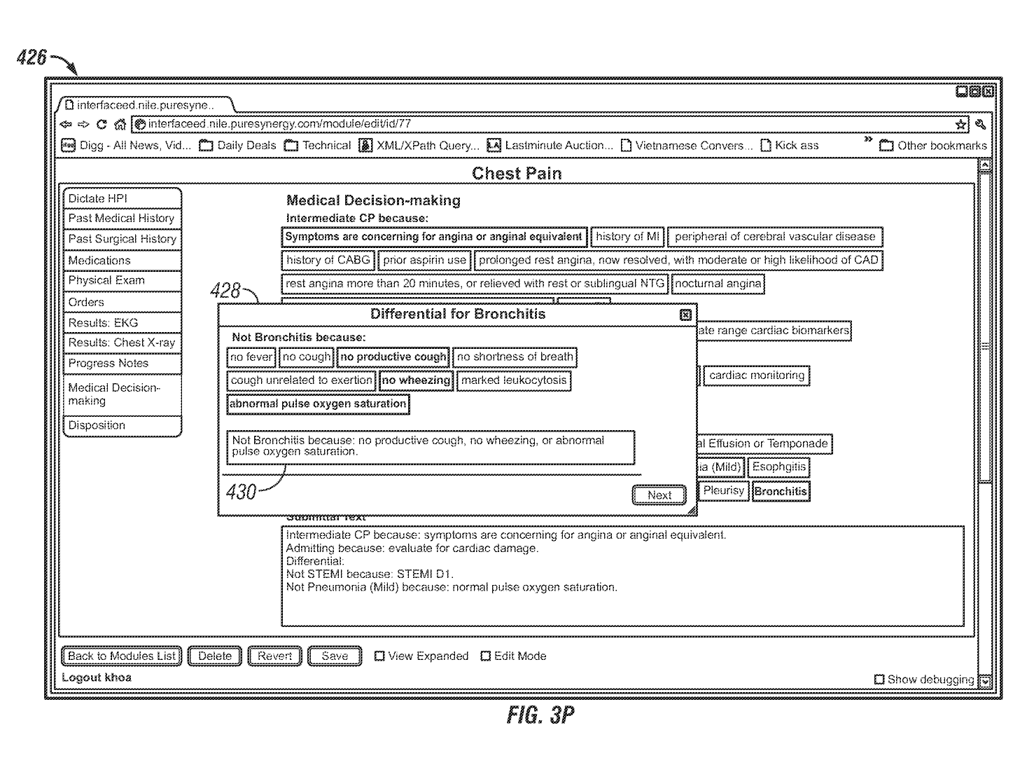

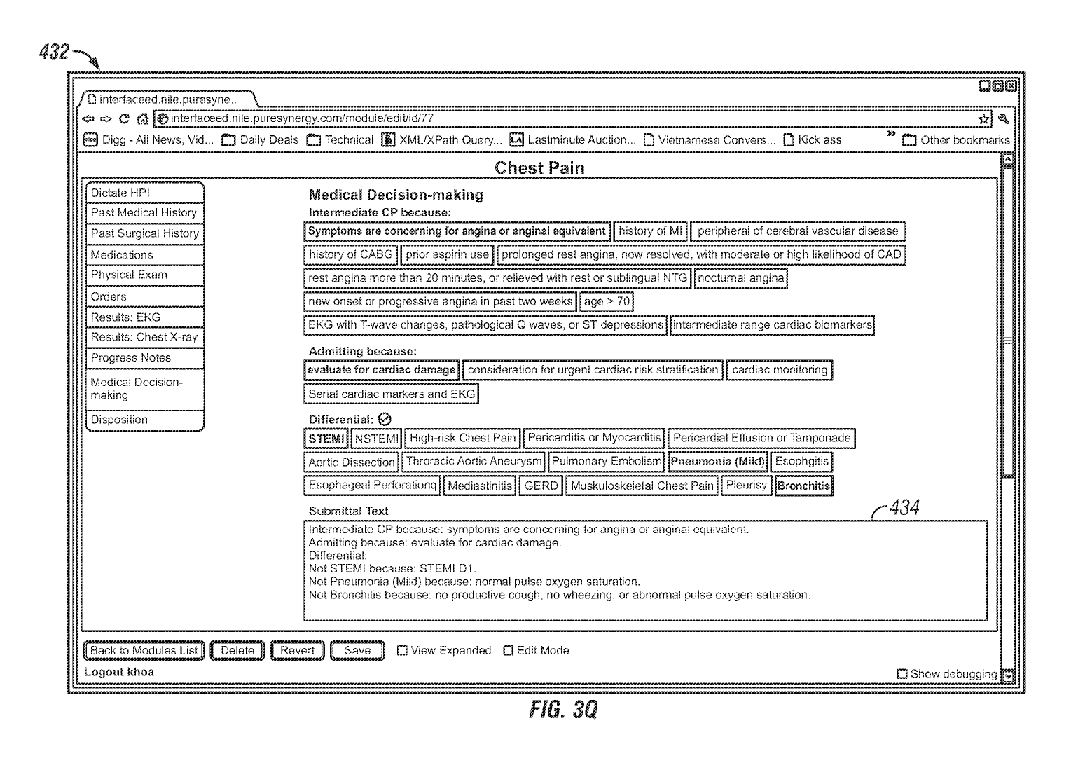

In certain embodiments, each of the templates 134 includes a plurality of queries associated with a medical diagnosis, at least one suggested response to each of the queries, and an input field, for each of the queries, configured to receive an actual response for each of the queries. The suggested response is configured to be selected by the user for entry into each of the input fields. In certain embodiments, each template comprises a plurality of suggested responses to each query, and each of the suggested responses is configured to be selected by the user for entry into the input field. For example, one suggested response can be entered into each input field. In certain embodiments, the suggested responses are configured to be selected by the user for simultaneous entry into the input field. For example, several suggested responses can be entered into each input field. The input field is configured to receive any text entered by the user, and is not limited to receiving suggested responses selected by the user. For example, the user can click on the input field using a mouse and begin typing text of his/her choosing. Exemplary templates 134 are illustrated in FIGS. 3A, 3B, 3C, 3D, 3E, 3F, 3G, 3H, 3I, 3J, 3K, 3L, 3M, 3N, 3O, 3P, and 3Q and will be described in further detail with reference to FIGS. 3A, 3B, 3C, 3D, 3E, 3F, 3G, 3H, 3I, 3J, 3K, 3L, 3M, 3N, 3O, 3P, and 3Q.

In certain embodiments, each of the templates 134 is configured to include recently published information related to the medical diagnosis associated with the template. The recently published information can be retrieved, for example, from a medical database or website over the Internet (i.e., the wide area network 118). For example, if a selected template is associated with a diagnosis of chest pain, the selected template may include recent information (e.g., within the last 1, 2, 3, 6, or 12 months) on new surgical or drug treatments for chest pain.

In certain embodiments, the design of each of the templates 134 is configured to be customizable. For example, each template can be customized based on a user's most often used preferences, intuitive design choices, site or facility look (e.g., logos and colors), and best practices.

The processor 112 of the client 102 is configured to receive, from a user, an input identifying a medical diagnosis and provide a template to be displayed on the display device 114 based on the identified medical diagnosis. The template can either be provided from the local templates cache 106 of the local server 150, or over the wide area network 118 from the templates 134 stored on the remote server 130. The processor 112 of the client 102 is also configured to receive responses to the queries in the template (e.g., via input device 116) and generate a record (e.g., that includes both the queries from the template and the received responses). The client 102 may thereafter store the record in memory 104 and/or send the record to the local server 150 over the local area network 119. The local server 150 is configured to use the EMR interface 108 in its memory 152 to convert the record into a format compatible with an EMR system, such as an EMR system in communication with the local server 150 for storing patient records for patients at a healthcare facility associated with the user. The EMR interface 108 allows the record to further interface with different types of EMR systems, such as an EDIS system, such as by converting the completed template into an EMR for storage in an EMR system. Therefore, compatibility between the completed template and an EMR system is addressed by the use of an appropriately configured EMR interface 108.

In certain embodiments, the EMR interface 108 is configured to work with pre-existing EMRs (e.g., including EMR systems) through the use of existing interface mechanisms, including, but not limited to, Application Program Interfaces (API) for the EMRs and the EMRs' own graphical user interfaces. In such cases, the EMR interface 108 can be considered to make the disclosed system “universally applicable” or “universally compatible” with such pre-existing EMRs. Information may be provided from the EMR to the EMR interface 108 synchronously, asynchronously, serially, and/or in parallel with other processes. In embodiments of the disclosed system that use APIs, programmatic calls are generated by the EMR interface 108 that pass information to and from the EMR. Alternatively, if the provider does not want to use the disclosed system to enter information into the EMR, then the provider can enter information into the EMR directly, such as by disabling the EMR interface 108.

In certain embodiments of the disclosed system that use GUIs to interface with the underlying EMR, such as with underlying EMRs that do not provide APIs, the EMR interface 108 implements screen scraping. The screen scraping interaction includes reading the screen of the underlying EMR, determining how to interact with the read screen, and executing keyboard, mouse, and other input instructions to provide information to the underlying EMR screen automatically using the EMR interface 108. For example, in certain embodiments, the mouse instructions include cursor movements and clicks. The keyboard instructions include sending keystrokes, including shortcuts like cut and paste as allowed by an underlying operating system. In certain embodiments, the EMR interface 108 implements image processing and/or recognition. Image processing and/or recognition includes optically visualizing data on a tangible medium, recognizing the content of the data on the tangible medium, and converting it to provide information to the underlying EMR screen using the EMR interface 108. For example, in certain embodiments, optical character recognition may be applied to convert scanned images of handwritten, typewritten or printed text into machine-encoded text.

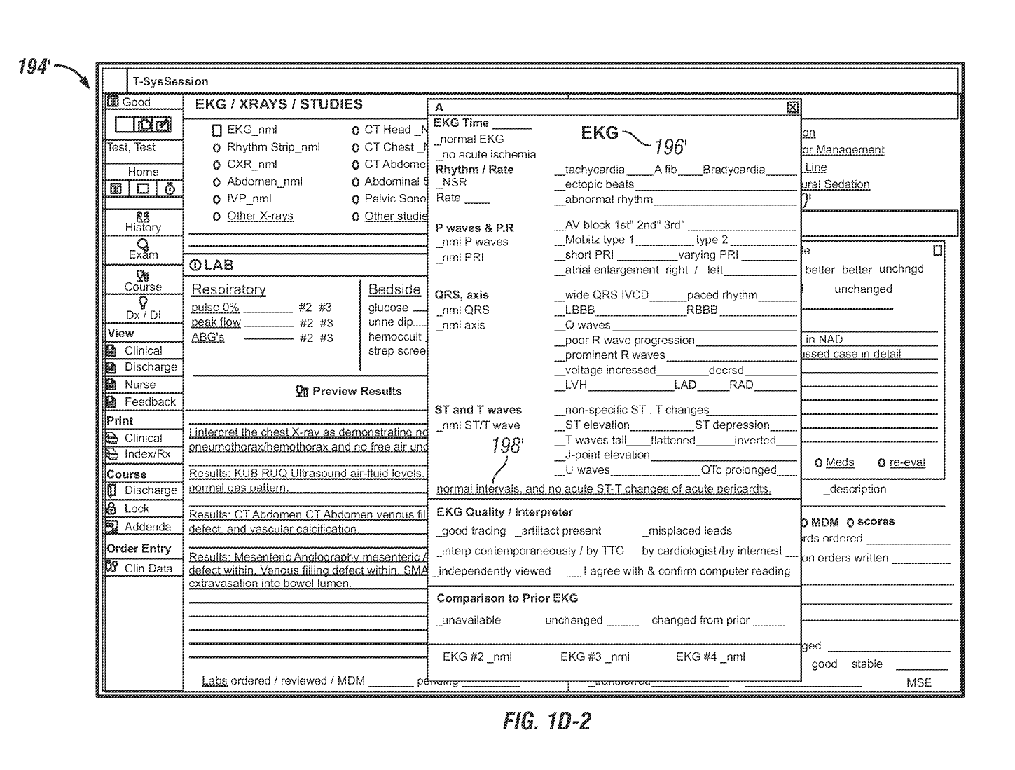

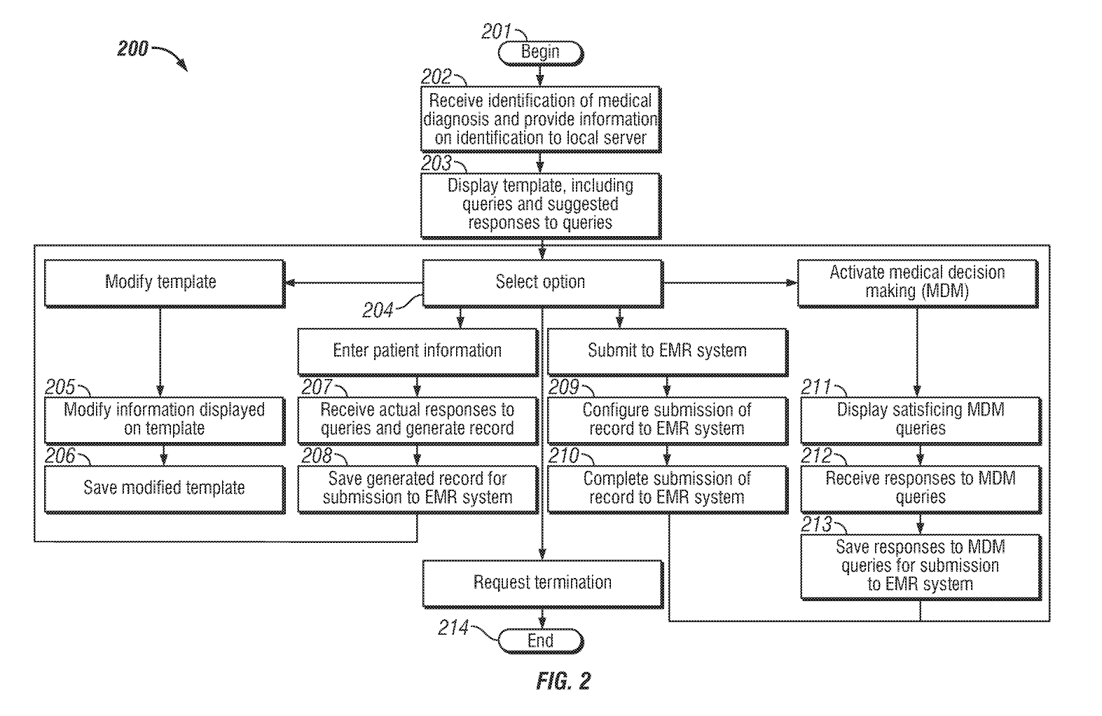

FIGS. 1B-1 and 1B-2 illustrate an exemplary screenshot 182 of a user interface for entering a history of patient information (HPI) using the disclosed system and an exemplary screenshot 182′ of a user interface for entering a HPI using a conventional EMR system. The EMR interface 108 is configured to read the screen 182′ of the underlying conventional EMR system and make associations with the disclosed system. For example, the EMR interface 108 is configured to associate the “Dictate HPI” query 184 of the disclosed system with the “HPI” entry point 184′ of the underlying EMR system. Similarly, the EMR interface 108 is configured to associate the “Dictate HPI” input field 186 of the disclosed system with the “Other History” input field 186′ of the underlying EMR system.

FIGS. 1C-1 and 1C-2 illustrate an exemplary screenshot 188 of a user interface for entering progress notes using the disclosed system and an exemplary screenshot 188′ of a user interface for entering progress notes using a conventional EMR system. The EMR interface 108 is configured to read the screen 188′ of the underlying conventional EMR system and make associations with the disclosed system. For example, the EMR interface 108 is configured to associate the “Progress Notes” query 190 of the disclosed system with the “Progress” entry point 190′ of the underlying EMR system. Similarly, the EMR interface 108 is configured to associate the “Progress Notes” input field 192 of the disclosed system with the input field 192′ of the underlying EMR system.

FIGS. 1D-1 and 1D-2 illustrate an exemplary screenshot 194 of a user interface for entering electrocardiogram (EKG) results using the disclosed system and an exemplary screenshot 194′ of a user interface for entering EKG results using a conventional EMR system. The EMR interface 108 is configured to read the screen 194′ of the underlying conventional EMR system and make associations with the disclosed system. For example, the EMR interface 108 is configured to associate the “Results: EKG” query 196 of the disclosed system with the “EKG” entry point 196′ of the underlying EMR system. Similarly, the EMR interface 108 is configured to associate the “Results: EKG” input field 198 of the disclosed system with the input field 198′ of the underlying EMR system.

In certain embodiments, the processor 150 of the remote server 130 is similarly configured to receive, from a user, and over the local area network 119 and the wide area network 118, an input identifying a medical diagnosis. In certain embodiments, the processor 150 of the remote server 130 is configured to provide a template to be displayed on the display device 114 of the client 102 based on the identified medical diagnosis. The template is provided from the templates 134 stored on the remote server 130. For example, if the appropriate template for the identified medical diagnosis is not available in the local templates cache 106, the appropriate template is provided from the templates 134 stored in the memory 132 of the remote server 130.

The architecture 100 of FIG. 1A is exemplary; other architectures are contemplated and can be used with the disclosure. For example, in certain embodiments, the EMR interface 108 can be stored in the memory 104 of the client 102, thereby allowing the client 102 to process the generated records. By way of another example, in certain embodiments, the EMR interface 108 and local templates cache 106 can be stored in the memory 104 of the client 102, thereby removing the need for the local server 150. In such cases, the communications module 102 of the client 102 can be directly connected to the communications module 138 of the remote server 130 over the wide area network 118.

FIG. 2 is an exemplary process 200 for providing information on a medical diagnosis using the architecture 100 of FIG. 1A. The process 200 begins from step 201 to step 202, where an identification of a medical diagnosis is received (e.g., at the client 102) and information on the identified medical diagnosis is provided (e.g., to the local server 150). In step 203, a template is displayed (e.g., from the local templates cache 106 and/or from local server 150) that is based on the identified medical diagnosis. In step 204, one of five options is selected.

If the user chooses to modify the template in step 204, then the modified template is displayed in step 205. At this step, the template can be modified 134 by the user editing the suggested responses (as will be described in more detail with reference to FIGS. 3I-3L). The modified template is saved (e.g., locally in the local templates cache 106 of the local server 150 and/or remotely along with the templates 134 on the remote server 130) in step 206. The process 200 then returns to step 204.

If the user chooses to enter patient information in step 204, then the process 200 proceeds to step 207 in which actual responses to queries in the template are received, and a record is generated. In step 208, the generated record is saved for later submission to the EMR system. The process 200 then returns to step 204.

If the user chooses to activate the Medical Decision Making process (e.g., Medical Decision Making queries) in step 204, then the process 200 proceeds to step 211. Medical Decision Making satisficing queries are displayed in step 211 and responses to the queries are received in step 212. As discussed herein, satisficing is a decision-making strategy that attempts to meet criteria for adequacy, rather than to identify an optimal solution. The responses are saved in step 213 for later submission to the EMR system. The process 200 then returns to step 204.

If the user chooses to submit a generated record (including responses to Medical Decision Making queries) to the EMR system in step 204, then the process 200 proceeds to step 209 in which the record submission is configured for the EMR system. For example, the user can choose to have the record submitted serially with other generated records (and/or individual response to queries), in parallel with other generated records, or manually. In step 201, the record is submitted to the EMR system using the EMR interface 108 based on the configuration of step 209. The process 200 then returns to step 204.

FIGS. 3A-3L are exemplary screenshots that illustrate various steps for providing information on a medical diagnosis using the architecture 100 of FIG. 1A. FIG. 3A is an exemplary screenshot 300, from display device 114, of a template associated with a diagnosis of chest pain 302. The template is likely to be displayed to a user, such as a healthcare provider, during or after the user has completed a meeting with a patient. The diagnosis 302 is identified in the template. The template also displays a plurality of suggested responses 304 and 306 to the query “Dictate HPI” 314. The “Dictate HPI” 314 query is one of a plurality of queries 315 that is included in a template. Each of the plurality of queries 315 for each template can be selectively chosen for display, such as by the provider. In certain embodiments, queries 315 are chosen for display if display of the queries will help the provider improve efficiency in entering patient information.

The suggested responses 304 and 306 to the “Dictate HPI” query 314 are divided into two categories 308 and 310 based on the content of the suggested responses 304 and 306. The categories are based on a previously provided response to the at least one query, a user-defined suggested response, facility policy, and a best practice. In the illustrated example, the suggested responses 304 for dictating a History of Present Illness (HPI) include that the illness is “not associated with” 308 the first category of suggested responses 304, and that the HPI is “neither exacerbated or relieved by” 310 the second category of suggested responses 306. The template also includes an input field 312 in which the actual response by the user will be entered.

FIG. 3B is an exemplary screenshot 320 of the template of FIG. 3A after a suggested response has been selected by the user. Specifically, the suggested response “SOB” 322, which stands for shortness of breath, has been selected by the user. After a suggested response 322 is selected by the user, such as by using a keyboard, mouse, pen, or voice command 116, the processor 112 of the client 102 is configured to enter the selected suggested response into the input field 312, as illustrated. The entry, illustrated as “Not associated with SOB,” is based on the selected suggested response “SOB” 322, but is more detailed than the selected suggested response 322. For example, instead of just entering the text “SOB” into the input field 312, the text “Not associated with SOB” is entered instead, thereby providing more information on what the selection of the suggested response 322 is intended to mean.

FIG. 3C is an exemplary screenshot 330 of the template of FIG. 3B after a plurality of suggested responses 324, 326, 328, 332, and 334 have been selected by the user. Specifically, the suggested responses “SOB” 322, “palpitations” 326, “nausea” 328, “position” 332, and “exertion” 334 have been selected by the user. As the suggested responses 324, 326, 328, 332, and 334 are selected by the user, the processor 112 of the client 102 is configured to enter the selected suggested responses 324, 326, 328, 332, and 334 together (or “simultaneously”) into the input field 312, as illustrated. The entry, illustrated as “Not associated with SOB, palpitations, or nausea. Neither exacerbated or relieved by position or exertion,” is based on the selected suggested responses 324, 326, 328, 332, and 334, but is more detailed than the selected suggested responses 324, 326, 328, 332, and 334, and is in a grammatical format that combines the selected plurality of suggested responses. For example, instead of just entering the text “SOB palpitations nausea position exertion” into the input field 312, the text “Not associated with SOB, palpitations, or nausea. Neither exacerbated or relieved by position or exertion,” is entered instead, thereby providing more information on what the selection of the suggested responses 324, 326, 328, 332, and 334 is intended to mean.

FIG. 3D is an exemplary screenshot 340 of the template of FIGS. 3A-3C after a plurality of suggested responses 344 and 346 have been selected by the user in response to a query 342 regarding past medical history. Specifically, the suggested responses “no prior CVA” 344 and “no connective tissue disease” 346 have been selected by the user. As the suggested responses 344 and 346 are selected by the user, the processor 112 of the client 102 is configured to enter the selected suggested responses 344 and 346 simultaneously into the input field 312, as illustrated. The entry, illustrated as “Previous medical conditions: no prior CVA and no connective tissue disease,” is based on the selected suggested responses 344 and 346, but is more detailed than the selected suggested responses 344 and 346, and is in a grammatical format that combines the selected plurality of suggested responses 344 and 346.

FIG. 3E is an exemplary screenshot 350 of the template of FIGS. 3A-3D after a plurality of suggested responses 354, 356, 358, and 360 have been selected by the user in response to a query 352 regarding past surgical history and after the user has provided his own input in response to the query 352. Specifically, the suggested responses “no CABG” 354, “no PCI” 356, “no prior aorta surgeries” 358, and “no prior thoracic surgeries” 360 have been selected by the user. As the suggested responses 354, 356, 358, and 360 are selected by the user, the processor 112 of the client 102 is configured to enter the selected suggested responses 354, 356, 358, and 360 simultaneously into the input field 312, as illustrated. The entry, illustrated as “Past surgeries: no CABG, no PCI, no prior aorta surgeries, and no prior thoracic surgeries,” is based on the selected suggested responses 354, 356, 358, and 360, but is more detailed than the selected suggested responses 354, 356, 358, and 360, and is in a grammatical format that combines the selected plurality of suggested responses 354, 356, 358, and 360. The input field 312 also includes text entered manually by the user, for example, using a keyboard, namely the text “Patient reported he had appendix removed” 362.

FIG. 3F is an exemplary screenshot 370 of the template of FIGS. 3A-3E after a patient's physical exam information is entered, in response to a physical exam type query 372, into the input field 312 using suggested responses. Specifically, the suggested responses “brief” 372 for a general exam, “detailed” 374 for a HEENT exam, “detailed” 376 for a neck exam, “brief” 380 for a pulmonary exam, “detailed” 378 for a neurological exam, and “brief” 382 for a heme and lymph exam, have been selected by the user. As the suggested responses 372, 374, 376, 378, 380, and 382 are selected by the user, the processor 112 of the client 102 is configured to enter the selected suggested responses 372, 374, 376, 378, 380, and 382 simultaneously into the input field 312, as illustrated. The entry reflects more detailed information for each exam than whether the exam was brief or detailed. For example, the brief 372 entry for the general exam states “General: nontoxic, no acute distress, interacting appropriately,” while the detailed 374 entry for the HEENT exam states “HEENT: atraumatic, pharynx normal, moist mucous membranes, no tonsillar exudates, no drooling, TMs benign bilaterally, conjuctiva without injection or edudate, no rhinorhea.”

FIG. 3G is an exemplary screenshot 384 of the template of FIGS. 3A-3F in which a facility's best practices are identified in the suggested responses. Specifically, a suggested response 386 is provided in the template that is based on the best practices of the user's healthcare facility. As the suggested response 386 is selected by the user, the processor 112 of the client 102 is configured to enter the selected suggested responses 386 into the input field 312, thereby reflecting the facility's best practices.

FIG. 3H is an exemplary screenshot 388 of the template of FIGS. 3A-3G in which a suggested response 390, after entered into the input field 312, is configured to be edited. Specifically, the “[Dr. Admission]” portion of the suggested response 390 is configured to be edited in the input field 312 by selecting that portion (e.g., with a mouse click) and typing text (e.g., the admitting doctor's name) to replace that text.

FIG. 3I is an exemplary screenshot 392 of the templates of FIGS. 3A-3H after an editing mode has been entered. The editing mode is entered by the selecting the “Edit Mode” check box. Editing mode allows the template to be modified by a user, such as a healthcare provider or a system administrator. In editing mode, two buttons 394 and 395 are displayed, a subtraction button 394 represented by a subtraction sign, and an addition button 395 represented by an addition sign. Selection of the subtraction button 394 (e.g., by clicking the button with a mouse) removes an associated suggested response, and selection of the addition button 395 adds a new suggested response 398, as illustrated in the screenshot 396 of FIG. 3J. This feature may be used, for example, when a user finds that a suggested response he/she uses in responding to a query for a medical diagnosis is not present in the template. The new suggested response 398 can be edited as illustrated in the screenshot 400 of FIG. 3K, wherein the new suggested response 402 has been edited to read “Patient checked out AMA.” Thereafter, when edit mode is exited, as illustrated in the screenshot 404 of FIG. 3L, the newly added suggested response 406, upon selection, causes certain text, namely “Patient is appropriate for outpatient management: Patient checked out AMA,” to be entered into the input field 312.

FIG. 3M is an exemplary screenshot 410 of a Medical Decision Making (MDM) query. In certain embodiments, each template includes a MDM query. The MDM query is configured to provide criteria for satisficing the provider's selected diagnosis. The satisficing criteria facilitate the consideration and exclusion of competing diagnoses (e.g., diagnoses that would replace the selected diagnosis). In certain embodiments, the MDM query of each template is not required to be displayed to the provider. In certain embodiments where the MDM query is displayed, the MDM query required to be completed by the provider. Activation of the MDM query facilitates the provider in both documenting the decision making process for the selected diagnosis, and improving the accuracy of the selected diagnosis, thereby improving patient care. The MDM interface prompts a provider to provide positive reasons 412 to support his/her diagnosis/diagnoses 414. In certain embodiments, the provider also selects from a list of competing diagnoses in a “Differential” section 416 that may be eliminated from consideration for the patient complaint.

FIGS. 3N, 3O, and 3P are exemplary screenshots 418, 422, and 426 of prompts 420, 424, and 428 displayed in response to the eliminated diagnoses selected in the Differential section 416 of FIG. 3M. In certain embodiments, for each eliminated diagnosis that is selected, a prompt 420, 424, and 428 is displayed for the provider to enter reasons why the provider selected the diagnosis to be eliminated. In FIG. 3N, the prompt 420 is for the eliminated diagnosis “STEMI.” In FIG. 3O, the prompt 424 is for the eliminated diagnosis “Pneumonia (mild).” In FIG. 3P, the prompt 426 is for the eliminated diagnosis “bronchitis.” As discussed above, after a suggested response 428 is selected by the user (e.g., provider), such as by using a keyboard, mouse, pen, or voice command, the processor 112 of the client 102 is configured to enter text indicative of the selected suggested response into an associated input field 430, as illustrated.

FIG. 3Q is an exemplary screenshot 432 of the MDM interface of FIG. 3M after text indicative of the provider's selections have been entered into an associated input field 434. Specifically, the text in the associated input field 434 includes a description of the eliminated diagnoses that were selected by provider after prompting by the disclosed system.

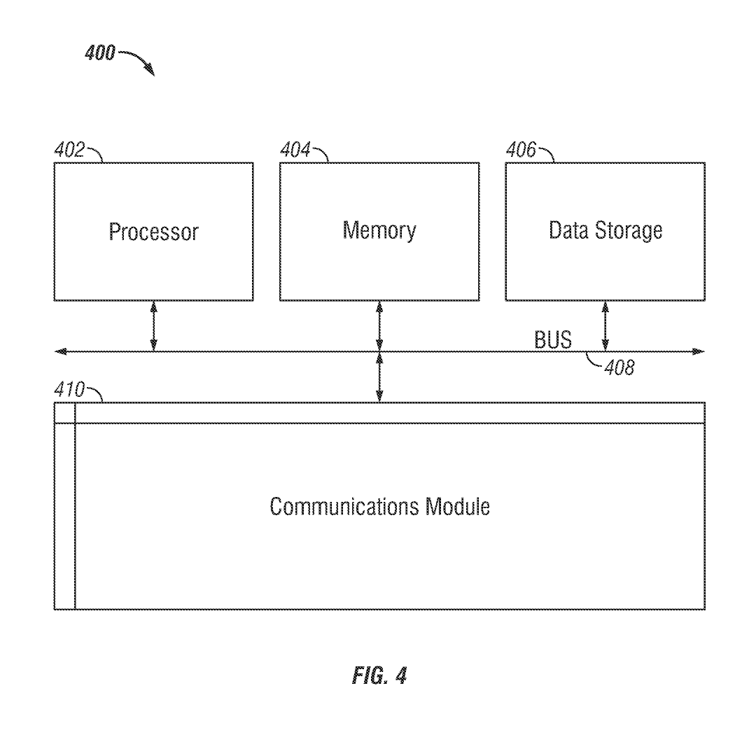

FIG. 4 is a block diagram illustrating an example of a computer system 450 with which the client 102, the local server 150, or the remote server 130 illustrated in FIG. 1A can be implemented. In certain embodiments, the computer system 450 may be implemented using software, hardware, or a combination of both, either in a dedicated server, or integrated into another entity, or distributed across multiple entities.

Computer system 450 (e.g., client 102, the local server 150, or the remote server 130 from FIG. 1A) includes a bus 458 or other communication mechanism for communicating information, and a processor 452 (e.g., processor 112, 154, or 136 from FIG. 1A) coupled with bus 458 for processing information. By way of example, the computer system 450 may be implemented with one or more processors 452. Processor 452 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information. Computer system 450 also includes a memory 454 (e.g., memory 104, 152, or 132 from FIG. 1A), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 458 for storing information and instructions to be executed by processor 452. The instructions may be implemented according to any method well known to those of skill in the art, including, but not limited to, computer languages such as data-oriented languages (e.g., SQL, dBase), system languages (e.g., C, Objective-C, C++, Assembly), architectural languages (e.g., Java), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 404 may also be used for storing temporary variable or other intermediate information during execution of instructions to be executed by processor 402. Computer system 450 further includes a data storage device 456 such as a magnetic disk or optical disk, coupled to bus 458 for storing information and instructions. Computer system 450 may be coupled via communications module 460 (e.g., communications module 110, 156, or 138 from FIG. 1A) to various devices (not illustrated). The communications module 410 can be any input/output module. In certain embodiments not illustrated, the communications module 410 is configured to connect to a plurality of devices, such as an input device (e.g., input device 116 from FIG. 1A) and/or a display device (e.g., display device 114 from FIG. 1A).

According to one aspect of the present disclosure, the client 102, the local server 150, or the remote server 130 can be implemented using a computer system 450 in response to processor 452 executing one or more sequences of one or more instructions contained in memory 454. Such instructions may be read into memory 454 from another machine-readable medium, such as data storage device 456. Execution of the sequences of instructions contained in main memory 454 causes processor 452 to perform the process steps described herein. One or more processors in a multi-processing arrangement may also be employed to execute the sequences of instructions contained in memory 454. In alternative embodiments, hard-wired circuitry may be used in place of or in combination with software instructions to implement various embodiments of the present disclosure. Thus, embodiments of the present disclosure are not limited to any specific combination of hardware circuitry and software.

The term “machine-readable medium” as used herein refers to any medium or media that participates in providing instructions to processor 452 for execution. Such a medium may take many forms, including, but not limited to, non-volatile media, volatile media, and transmission media. Non-volatile media include, for example, optical or magnetic disks, such as data storage device 456. Volatile media include dynamic memory, such as memory 454. Transmission media include coaxial cables, copper wire, and fiber optics, including the wires that comprise bus 458. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read.

The disclosed systems and methods provides a user interface for providing information on a medical diagnosis that addresses key issues in prior art EMRs that reduce productivity, change work flow, produce less useful clinical documentation, and create the tools to improve the end users' experience through a more robust interface. The disclosed systems and methods use a diagnosis driven protocol to provide a user interface that is matched to a healthcare provider's cognitive process, which results in a substantial increase in workflow efficiency. In certain embodiments, the disclosed systems and methods result in an improved interaction between healthcare providers and EMRs, thereby creating workplace advantages and efficiencies by improving work flow efficiencies in medical departments and professional office settings, increasing professional reimbursement, decreasing time devoted directly to electronic documentation in the order of hours a day, improving quality of work life and job satisfaction, resulting in reduced staff turnover and greater group stability, increasing patient satisfaction, and increasing quality of care delivery and decreasing medical liability.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

What is claimed is:

1. A method of providing information on a medical diagnosis, comprising:
receiving, from a user, an input identifying the medical diagnosis;
requesting, from a server, a template based on the identified medical diagnosis the template comprising:
at least one query associated with the identified medical diagnosis;
at least one suggested response to the at least one query; and
an input field configured to receive the at least one suggested response to the at least one query,
wherein the at least one suggested response is configured to be selected by the user for entry into the input field;
receiving, from the server, the template;
displaying, on a display device, the template to the user; and
generating a record based on the template and the at least one suggested response, by the user, to the at least one query.

2. The method of claim 1, wherein the input field is configured to receive text entered by the user.

3. The method of claim 2, wherein the text entered by the user is entered into a target EMR via at least one of an exposed Application Programming Interface layer, mouse/keyboard input, and image processing/recognition.

4. The method of claim 1, wherein the at least one suggested response is based on at least one of a previously provided response to the at least one query, a user-defined suggested response, a facility policy, and a best practice.

5. The method of claim 1, wherein entry of the at least one suggested response into the input field comprises entering a response into the input field that is based on the at least one suggested response and is more detailed than the at least one suggested response.

6. The method of claim 1, wherein the template comprises:
a plurality of queries associated with the identified medical diagnosis;
at least one suggested response to each of the plurality of queries; and
an input field, for each of the plurality of queries, configured to receive the at least one suggested response for each of the plurality of queries,
wherein the at least one suggested response for each of the plurality of queries is configured to be selected by the user for entry into the respective input field for each of the plurality of queries.

7. The method of claim 1, wherein the template comprises a plurality of suggested responses to the at least one query, and wherein each of the plurality of suggested responses is configured to be selected by the user for entry into the input field.

8. The method of claim 7, wherein each of the plurality of suggested responses is categorized within one of at least two of a previously provided response to the at least one query, a user-defined suggested response, facility policy, and a best practice.

9. The method of claim 7, wherein each of the plurality of suggested responses is configured to be selected by the user for simultaneous entry into the input field.

10. The method of claim 9, wherein simultaneous entry of the selected plurality of suggested responses into the input field comprises entering a response into the input field that is based on the selected plurality of suggested responses, is more detailed than the selected plurality of suggested responses, and is in a grammatical format that combines the selected plurality of suggested responses.

11. The method of claim 1, wherein the provided template is selected from a plurality of templates, each of the plurality of templates being associated with at least one medical diagnosis.

12. The method of claim 1, wherein the provided template further comprises published information related to the identified medical diagnosis.

13. The method of claim 1, further comprising converting the record into a format compatible with an electronic medical record system.

14. A system for providing information on a medical diagnosis, comprising:
a processor configured to request, from a server, a template based on the medical diagnosis, and to receive the template from the server;
a memory comprising the template, the template comprising:
at least one query associated with the medical diagnosis;
at least one suggested response to the at least one query; and
an input field configured to receive the at least one suggested response to the at least one query,
wherein the at least one suggested response is configured to be selected by a user for entry into the input field,
wherein the processor is further configured to receive, from the user, an input identifying the medical diagnosis, to provide the template based on the identified medical diagnosis, and to generate a record based on the template and the at least one suggested response, by the user, to the at least one query.

15. The system of claim 14, wherein the input field is configured to receive text entered by the user.

16. The system of claim 14, wherein the at least one suggested response is based on at least one of a previously provided response to the at least one query, a user-defined suggested response, a facility policy, and a best practice.

17. The system of claim 14, wherein the template comprises:
a plurality of queries associated with the identified medical diagnosis;
at least one suggested response to each of the plurality of queries; and
an input field, for each of the plurality of queries, configured to receive the at least one suggested response for each of the plurality of queries,
wherein the at least one suggested response for each of the plurality of queries is configured to be selected by the user for entry into the respective input field for each of the plurality of queries.

18. The system of claim 14, wherein the template comprises a plurality of suggested responses to the at least one query, and wherein each of the plurality of suggested responses is configured to be selected by the user for entry into the input field.

19. The system of claim 18, wherein each of the plurality of suggested responses is configured to be selected by the user for simultaneous entry into the input field.

20. The system of claim 19, wherein simultaneous entry of the selected plurality of suggested responses into the input field comprises entering a response into the input field that is based on the selected plurality of suggested responses, is more detailed than the selected plurality of suggested responses, and is in a grammatical format that combines the selected plurality of suggested responses.

21. A machine-readable medium comprising machine-readable instructions for causing a processor to execute a method of providing information on a medical diagnosis, comprising:
receiving, from a user, an input identifying the medical diagnosis;
requesting, from a server, a template based on the identified medical diagnosis the template comprising:
at least one query associated with the identified medical diagnosis;
at least one suggested response to the at least one query; and
an input field configured to receive the at least one suggested response to the at least one query,
wherein the at least one suggested response is configured to be selected by the user for entry into the input field;
receiving, from the server, the template;
displaying, on a display device, the template to the user; and
generating a record based on the template and the at least one suggested response, by the user, to the at least one query.

22. A system for providing information on a medical diagnosis, comprising:
a processor configured to receive, from a client, a request for a template based on the medical diagnosis;
a memory comprising the template, the template comprising:
at least one query associated with the medical diagnosis;
at least one suggested response to the at least one query; and
an input field configured to receive an actual response to the at least one query,
wherein the at least one suggested response is configured to be selected by a user for entry into the input field,
wherein the processor is further configured to provide, to the client, the template from the memory.

23. A method of providing information on a medical diagnosis, comprising:
receiving, from a client, a request for a template based on the medical diagnosis;
retrieving, from a memory, the template, the template comprising:
at least one query associated with the identified medical diagnosis;
at least one suggested response to the at least one query; and
an input field configured to receive the at least one suggested response to the at least one query,
wherein the at least one suggested response is configured to be selected by the user for entry into the input field; and
providing, to the client, the template.

24. A machine-readable medium comprising machine-readable instructions for causing a processor to execute a method of providing information on a medical diagnosis, comprising:
receiving, from a client, a request for a template based on the medical diagnosis;
retrieving, from a memory, the template, the template comprising:
at least one query associated with the identified medical diagnosis;
at least one suggested response to the at least one query; and
an input field configured to receive the at least one suggested response to the at least one query,
wherein the at least one suggested response is configured to be selected by the user for entry into the input field; and
providing, to the client, the template.

25. A method of confirming a medical diagnosis using a graphical user interface, comprising:
receiving an input selecting a first medical diagnosis as a cause of a patient complaint;
displaying, on a display device, a template based on the first medical diagnosis, the template comprising:
a first query associated with the first medical diagnosis;
a suggested response to the first query;
a first input field configured to receive the suggested response to the first query;
a second query associated with a second medical diagnosis, wherein the second medical diagnosis is an alternative to the first medical diagnosis;
a suggested response to the second query; and
a second input field configured to receive the suggested response to the second query,
wherein the suggested response to the first query is configured to be selected by the user for entry into the first input field and the suggested response to the second query is configured to be selected by the user for entry into the second input field; and
generating a record based on the template and a suggested response received by the first input field and a suggested response received by the second input field.
